(19) United States
(12) Plant Patent Application Publication
(10) Pub. No.: US 2012/0185979 P1
(43) Pub. Date: Jul. 19, 2012

(54) Clematis Plant Named 'Beautiful Bride'

(50) ;

(76) Inventor: Szczepan Witold Marczynski, Pruszkow (PL)

(21) Appl. No.: 12/930,893

(22) Filed: Jan. 19, 2011

(51) Int. Cl. A01H 5/00

(52) U.S. Cl. PLT/228

(57) ABSTRACT

A new and distinct variety of clematis plant named ‘Beautiful Bride’ particularly distinguished by its large, white, star-shaped flowers and long, pointed tepals, is disclosed.

### GENUS AND SPECIES

Clematis sp.

### VARIETY DENOMINATION

‘Beautiful Bride’

### BACKGROUND OF THE NEW PLANT

The present invention relates to a new and distinct clematis variety designated ‘Beautiful Bride’ and botanically known as Clematis sp. This new clematis variety was discovered in Pruszków, Poland and originated from a cross between an open pollination between the female clematis plant ‘Mrs. Cholmondeley’ (unpatented) and unknown clematis pollen parents (unpatented). ‘Beautiful Bride’ was subsequently asexually propagated and underwent further testing in Pruszków, Poland. The present invention has been found to be stable and reproduce true to type through successive asexual propagations via softwood cuttings.

### SUMMARY OF THE INVENTION

The following are the most outstanding and distinguishing characteristics of this new variety when grown under normal horticultural practices in Pruszków, Poland:

1. Large, white flowers;

2. Star-shaped flowers; and

3. Long, pointed tepals.

### PLANT BREEDERS RIGHTS

Plant Breeders Rights have been applied for in Poland on Dec. 15, 2009. ‘Beautiful Bride’ has not been sold or made publicly available more than one prior to the filing date of this application.

### DESCRIPTION OF THE PHOTOGRAPHS

This new clematis plant is illustrated by the accompanying photographs which show the overall plant habit, buds, and flowers. The colors shown are as true as can be reasonably obtained by conventional photographic procedures. The photographs are of plants that are 3 years old.

FIG. 1 shows the overall plant habit.

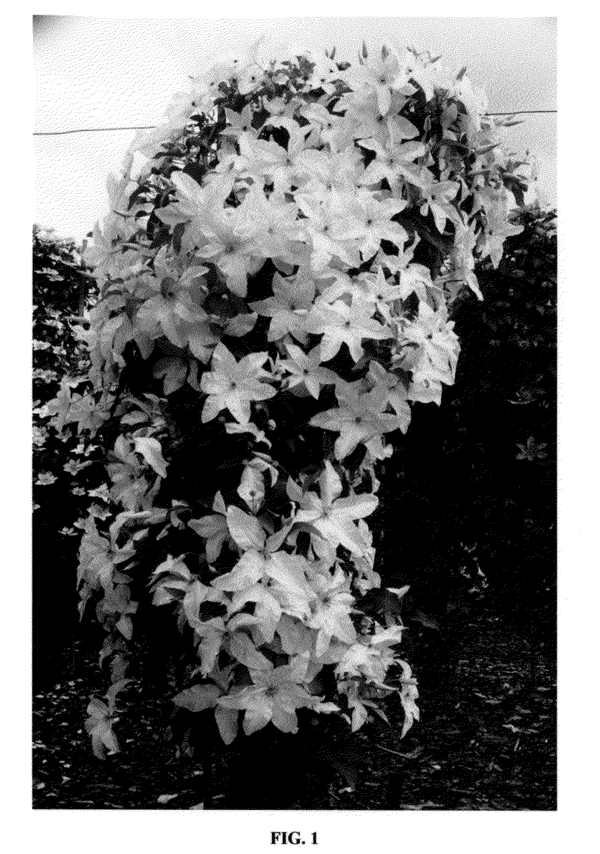

FIG. 2 shows a close up of the buds and flowers.

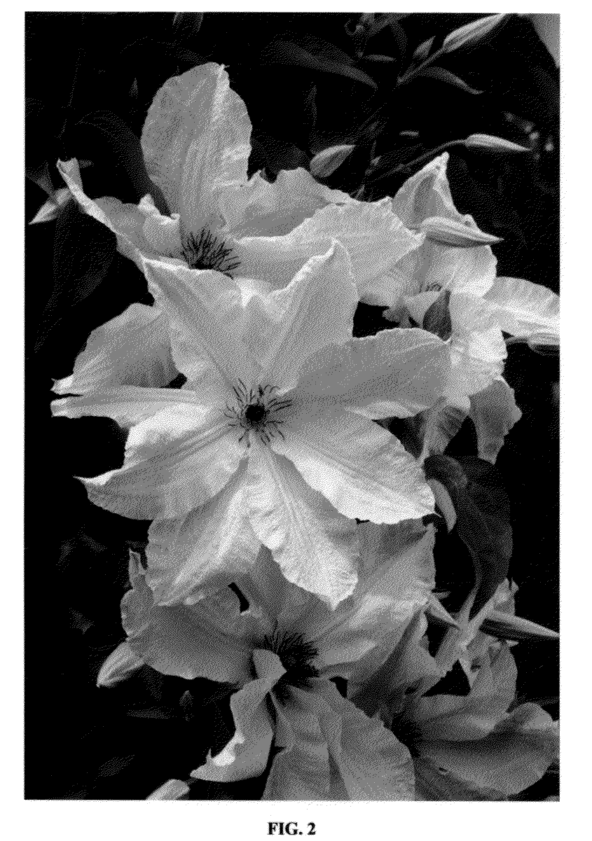

### DESCRIPTION OF THE NEW VARIETY

The following description of ‘Beautiful Bride’ is based on observations taken on three year old plants in Pruszków, Poland. This description is in accordance with UPOV terminology. Color designations, color descriptions, and other phenotypical descriptions may deviate from the stated values and descriptions depending upon variation in environmental, seasonal, climatic, and cultural conditions. ‘Beautiful Bride’ has not been observed under all possible environmental conditions. Color references are primarily to the R.H.S. Colour Chart of The Royal Horticultural Society of London (R.H.S.) (2001).

### DETAILED BOTANICAL DESCRIPTION

Classification:
   Family.—Ranunculaceae.
   Botanical.—clematis sp.
   Common name.—Clematis.
   Variety name.—‘Beautiful Bride’.
Propagation:
   Type.—By stem, softwood cuttings.
   Time to produce a rooted young plant, summer.—12 weeks.
   Root description.—Bundle root system.
   Rooting habit.—Adventitious roots developing from cambial callus.
Plant description:
   Form.—Climber.
   Plant habit.—The leafstalks or petioles wrap around convenient supports and help the plant climb.
   Plant height.—200.0 cm to 250.0 cm.
   Plant width.—100.0 cm.
Branching:
   Lateral branch.—Length: 20.0 cm to 30.0 cm Diameter: 0.2 cm to 0.3 cm Internode length: 4.0 cm to 8.0 cm Texture: Many-ribbed, downy hairs on young shoots Color: Immature: RHS 144B, early in season RHS 58A Mature: RHS 166A.
Foliage description:
   Arrangement.—Opposite.
   Type.—Simple and ternate.
   Length.—Simple 15.0 cm to 17.0 cm, ternate 16.0 cm to 22.0 cm.
   Width.—Simple 4.5 cm, ternate 15.0 cm to 18.0 cm.
   Shape.—Immature leaf is lanceolate, mature leaf is pointed-ovate.
   Apex.—Taper-pointed.
   Base.—Narrowed.
   Margin.—Entire; when immature, downy hairs are present.
   Texture.—Upper surface: Smooth, matt, main vein is concave Lower surface: Smooth, glossy, main vein is convex.
   Venation pattern.—Net pattern.
   Venation color.—Pale, grey-green.
   Foliage color.—Immature (both surfaces): RHS 143C Mature (both surfaces): RHS 144B.
   Terminal leaflet.—Length: 8.0 cm to 10.0 cm Width: 2.5 cm to 4.5 cm Shape: Pointed-ovate Apex: Taper-pointed Base: Narrowed Margin: Entire; when immature, downy hairs are present Texture: Upper surface: Smooth, matt, main vein is concave Lower surface: Smooth, glossy, main vein is convex Venation pattern: Net pattern Foliage color: Immature (both surfaces): RHS 143C Mature (both surfaces): RHS 144B.
   Lateral leaflets.—Length: 8.0 cm to 10.0 cm Width: 2.5 cm to 4.5 cm Shape: Pointed-ovate Apex: Taper-pointed Base: Narrowed Margin: Entire; when immature, downy hairs are present Texture: Upper surface: Smooth, matte, main vein is concave Lower surface: Smooth, glossy, main vein is convex Venation pattern: Net pattern Foliage color: Immature (both surfaces): RHS 143C Mature (both surfaces): RHS 144B.
   Petiole.—Length: 2.0 cm to 4.0 cm Diameter: 0.1 cm to 0.2 cm Color (both surfaces): RHS 59C Texture: Has downy hairs.
Flower description:
   Inflorescence type.—Compound cymes.
   Flower arrangement and shape.—Flat or flattish, star-shaped, upward- or outward-facing.
   Number of flowers.—In cymes, 5 to 11; on the plant, over 50.
   Flower aspect.—Upward- or outward-facing.
   Natural flowering season.—Late May on previous year's growth, to August on current year's vines.
   Flower longevity on the plant.—7 to 10 days.
   Fragrance.—Absent.
   Flower buds.—Length: 6.0 cm to 8.0 cm Diameter: 1.5 cm to 2.0 cm Shape: Long, pointed, downy Color: RHS 145D.
   Flowers.—Diameter: 18.0 cm to 28.0 cm Depth (height): 1.0 cm to 4.0 cm.
   Tepals.—Quantity and arrangement: 6 and overlapping Length: 9.0 cm to 13.0 cm Width: 4.5 cm to 6.0 cm Shape: Narrow elliptic to broad lanceolate and ribby Apex: Long-pointed Margin: Wavy Texture: Upper surface: 3 ribs concave, surface slightly wrinkled Lower surface: 3 ribs convex, surface slightly wrinkled Color when opening: Upper surface: White (no corresponding R.H.S. number) Lower surface: White with RHS 157C bar on ribs Color when fully open: Upper surface: White (no corresponding R.H.S. number), sometimes with a very light bar of RHS 62D Lower surface: White with RHS 157C bar on ribs.
   Peduncles.—Length: 2.0 cm to 6.0 cm Diameter: 0.3 cm to 0.4 cm Texture: Downy hairs present Color: RHS 144C, with RHS 64C.
Reproductive organs:
   Stamens.—Quantity per flower: About 100.
   Filament length.—1.0 cm to 1.5 cm.
   Filament color.—Lemon-colored.
   Anther shape.—Elongated.
   Anther length.—0.5 cm.
   Anther color.—Connectives are purple; anthers are white.
   Pollen amount.—Sparse.
   Pollen color.—Grey-white.
   Pistils.—Quantity per flower: 15 to 30 Length: 2.0 cm.
   Stigma color.—Lemon-colored.
   Style length.—1.0 cm.
   Style color.—Lemon-colored.
   Ovary color.—Lemon-colored.
Seed production: Many seed/fruit, fruit-heads persistent.
Disease resistance: Typical of species.

### COMPARISON WITH PARENTAL AND COMMERCIAL VARIETIES

When ‘Beautiful Bride’ is compared with the female parent ‘Mrs. Cholmondeley’ (unpatented), ‘Beautiful Bride’ has white flowers, a flower diameter of 18.0 cm to 28.0 cm, and long-pointed tepals, while ‘Mrs. Cholmondeley’ has blue flowers, a flower diameter of 15.0 cm to 20.0 cm, and mucronate-tipped tepals.

When ‘Beautiful Bride’ is compared to the commercial variety ‘Marie Boiselot’ (unpatented), ‘Beautiful Bride’ has six star-shaped and narrow tepals and a flower diameter of 18.0 cm to 28.0 cm, while ‘Marie Boiselot’ has eight broad and blunt tepals and a flower diameter of 15.0 cm to 20.0 cm.

I claim:

1. A new and distinct variety of clematis plant named ‘Beautiful Bride’ as described and shown herein.
